(19) United States
(12) Patent Application Publication
(10) Pub. No.: US 2016/0361393 A1
(43) Pub. Date: Dec. 15, 2016

(54) TOPICAL STING/BITE FORMULATION AND METHOD OF USE

(71) Applicant: BioStat Research, LLC, Orlando, FL (US)

(72) Inventors: William Fletcher Huggins, Leesburg, FL (US); Joanne S. Walter, Blue Ridge, GA (US)

(73) Assignee: BIOSTAT RESEARCH, LLC, Orlando, FL (US)

(21) Appl. No.: 14/737,119

(22) Filed: Jun. 11, 2015

(51) Int. Cl. A61K 38/48; A61K 36/886; A61K 47/46; A61K 36/68; A61K 36/53; A61K 36/28

(52) U.S. Cl. CPC A61K 38/4873; A61K 36/53; A61K 36/28; A61K 47/46; A61K 36/68; A61K 36/886; C12Y 304/22033; C12Y 304/22032

(57) ABSTRACT

A topical sting/bite treatment compound and method of making same comprising combining effective amounts of: a natural enzyme; a natural abrasive; a natural pain reliever; a natural anti-inflammatory; and a natural anti-microbial agent. The topical sting/bite treatment compound can neutralize venom, debride the sting/bite wound site, relieve pain, reduce swelling and prevent infection.

### BACKGROUND OF THE INVENTION

Field of the Invention

The present invention relates to sting and bite treatment formulations and more particularly to natural sting and bite treatment formulations free of papain.

Description of Related Art

Stings and bites by organisms are often accompanied by venoms, toxins, apitoxins or other poisonous compounds (collectively, “venoms”) that can cause significant discomfort and swelling. The physiological issues resulting from a sting or itch can be mild or severe. In a 2003 interview with Discover magazine, Justin Schmidt, entomologist at the University of Arizona, compared wasps and other stinging insects to “hackers” manipulating the cellular mechanisms of their victims. While each sting differs in the exact formulation, the chemicals injected in many stings and bites perform a similar series of actions designed to generate immediate pain, often without any long-term physical damage.

Venom enzymes can soften and ultimately burst open the membranes of otherwise healthy cells. Some of these cells, invariably, will be nerve cells. The destruction of the cellular membranes gives rise to an interruption in the electrochemical signaling that nerve cells conduct causing the cells to fire-off random signals. This can cause not only pain but can also result in associated spasms, shaking and even shock. Some chemicals contained in various venoms restrict blood flow thereby maintaining the pain-causing venom localized in an undiluted form. Simultaneously, additional chemicals found in some venoms are released that can promote the pain-inducing venoms to migrate on to new cellular targets. Finally, released pheromones can operate to enrage other venom carrying organisms, inciting them to join-in or continue an attack.

Traditional treatments for bites and stings rarely consist of more than soapy water, topical antihistamines or general analgesics. The FDA has a number of recommendations to relieve the itching and pain caused by bites and stings. Oral over-the-counter (“OTC”) antihistamines can bring general itch relief caused by mild to moderate allergic reactions. Oral OTC analgesic drugs, such as aspirin, acetaminophen, ibuprofen and naproxen sodium, provide some relief of pain from bites and stings; however, such relief is limited in efficacy and duration.

Topical OTC drugs, applied directly to the skin, can provide some level of itch and pain relief. These products are often labeled as “external analgesics” or “topical analgesics” and contain ingredients such as hydrocortisone, pramoxine, and lidocaine. Other topical OTC treatments, labeled as “skin protectants” provide itch relief for insect bites and stings, containing ingredients such as colloidal oatmeal and sodium bicarbonate.

Past topical OTC treatments included the natural enzyme papain (papaya protease I) found in papaya. Papain is generally banned by the FDA for being contrary to consumers' health due to the number of serious adverse events associated with the use of products containing papain. Adverse events, such as, allergic reactions (hypersensitivity), hypotension (low blood pressure) and tachycardia (rapid heart rate) have been reported.

Another issue associated with stings and bites is infection. Secondary infections caused by bacteria beneath the fingernails can be even more dangerous than the venoms from the stings or bites. By neutralizing venoms and providing a natural antimicrobial, pain and itch can be eliminated. Thus reducing the risk of infection from scratching. With no residue and no smell, the formula disclosed herein is highly desirable for use in both the civilian and military markets. This disclosure describes a natural topical sting/bite treatment formulation and method free of papain.

### SUMMARY OF THE INVENTION

The present invention is directed to a topical sting/bite treatment. Disclosed herein is a topical formula comprising natural ingredients and abrasives that can operate to mildly debride the skin cells, rather than simply remove the surface layer of skin. Such debridement ensures a better stinger, or other venom carrier, removal and/or allows better penetration of a natural neutralizing agent for the venom.

The topical sting/bite treatment formulation disclosed herein can include a natural enzyme capable of breaking down proteins, a natural abrasive capable of debriding or exfoliating an area on the skin, a natural pain reliever capable of reducing the pain associated with the venom of a sting or bite, a natural anti-inflammatory agent for reducing the swelling associated with a sting or bite and a natural anti-microbial agent to inhibit infection.

As disclosed herein the formula can also comprise a moisturizer to moisturize the skin.

The formula can be delivered in gel form to eliminate unwanted sticky or oily residue.

As the formulation is applied to the skin with gentle pressure, the natural abrasives operate to help debride the sting site and assist in removing any remaining stinger or other foreign objects imbedded in the skin. The natural antimicrobial agent operates to assist in reducing the incidence of infection and the natural pain reliever and anti-inflammatory assist in reducing the incidence of pain and itch.

### DETAILED DESCRIPTION OF THE PREFERRED EMBODIMENTS

A description of the preferred embodiments of the present invention will now be presented.

Stopping the pain and itch associated with a sting or bite requires an opening of the pores, debridement of the site, neutralizing the venom and minimizing the risk of infection. The simple, water-based gel approach disclosed herein completes these steps simultaneously for immediate and lasting relief. The active ingredients of the topical sting/bite treatment described herein include: a natural enzyme, a natural abrasive, a natural pain reliever, a natural anti-inflammatory and a natural anti-microbial agent.

By way of example, natural enzymes, such as bromelain, found in pineapple juice and pineapple stems, can be used in the treatment formulation disclosed herein to assist with and contribute to the break-down of certain proteins found in certain venoms associated with stings and bites. Bromelain is a protease enzyme that can operate to separate bonded amino acids and other organic compounds contained within living cells.

Generally, amino acids join by forming peptide bonds, a link that connects one amino acid's amino group with the carboxyl group of another amino acid. When amino acids join through peptide bonds, they form proteins. These proteins then carry out numerous functions in the structure and operation of cells, tissues and organs.

Skin contains collagen which operates to maintain the stricture and firmness of the skin tissue. When venom is introduced into the skin, it often remains localized near the sting or bite until such time as it loses its potency or dissipates into the surrounding tissues. The collagen in the skin contains proteins created from bonded amino acids. Bromelain operates to separate the all-important peptide bonds that link the amino acids thus resulting in the skin losing its firmness and allowing a faster dissipation of the venom into surrounding tissues.

An additional benefit to the operation of the natural enzyme bromelain is its anti-inflammatory properties.

Natural abrasives can comprise natural husks and seeds including crushed walnut shells, apricot kernels, almond seeds, raspberry seeds, blackberry seeds and grape seeds. Natural abrasives can also include mineral-like abrasives such as silica, pumice, coral and the like. These abrasives can be used for mild debridement of the bite or sting site. Debridement operates to exfoliate the area to remove a stinger or other venom carrier in an effort to stop any continued or additional stinging. Of further benefit, the natural abrasives can be effective in exfoliating skin cells for a better transfer of the active ingredients of the topical sting/bite treatment formulation to the site of the bite or sting. Other natural exfoliants, such as alpha hydroxyl and beta hydroxyl acids, can also be used to assist in debridement.

The topical sting/bite treatment formulation disclosed herein further comprises a natural pain reliever. Natural pain relievers, such as, for example Holy Basil or Tulsi (Ocimum sanctum), ginger, turmeric, devil's claw (Harpagophytum procumbens), Birch leaf (Betula Lenta), Wintergreen oil, Capsaicin (Capsicum), gamma-linolenic acid (GLA), Aquamin, Arnica, Peppermint (Mentha piperita), Boswellia and the like can include analgesic compounds and can be used to alleviate the pain often associated with the venom contained in certain stings and bites. For example, some of the chemical constituents of Holy Basil or Tulsi include: oleanolic acid, ursolic acid, rosmarinic acid, eugenol, carvacrol, linalool, β-caryophyllene (about 8%), β-elemene (c. 11.0%), and germacrene D (about 2%). A variety of in vitro studies and animal studies have indicated some potential pharmacological properties of natural pain relievers and related extracts. Other natural pain relievers such as trypsin, Barbadensis miller aloe vera, burdock root extract, Dioscorea villosa root extract, Cynoglossum officinale leaf extract, jewelweed (Impatiens sp.), lemonbalm (Melissa officinalis), prickly pear (Opuntia sp.), beebalm (monarada didyma), bowman's root (Gillenia trifoliata) and willow bark (Salix spp.) can also be used as natural analgesics to reduce the pain associated with a bite or sting.

The topical sting/bite treatment formulation disclosed herein further comprises a natural anti-inflammatory agent to reduce the swelling often associated with a sting or bite. Natural anti-inflammatory agents can include compounds such as marigold (Calendula officinalis) extract, cloves, cinnamon, oregano, marjoram, sage, rosemary, tyme, turmeric, ginger, green tea, cat's claw, devil's claw, willow and the like. Calendula has both anti-inflammatory and astringent properties and can be applied to the skin to reduce inflammation and associated pain and to treat poorly healing wounds as well as certain ulcers. Calendula may cause an allergic reaction in people who are sensitive to the Asteraceae/Compositae family. Members of this family include ragweed, chrysanthemums, marigolds, and daisies. Salix spp. (willow bark) is another natural anti-inflammatory that can be used according to the disclosure set forth herein.

The topical sting/bite treatment formulation disclosed herein further comprises a Natural anti-microbial agent. Natural anti-microbial agents can include compounds such as Broadleaf or Greater Plantain (Plantago major) extract, Manuka honey, tea catechin, tea tree, garlic, zinc, vitamin A, cranberry, cinnamon, clove, guava, lemongrass thyme, eucalyptus, cumin, rosemary, mango, wormwood, tarragon, nutmeg, lavender, goldenseal root and the like. By way of example, Plantago major is one of the most abundant and widely distributed medicinal crops in the world. A preparation of the leaves can be applied to wounds, stings, and sores in order to facilitate healing and prevent infection. The active chemical constituents include: aucubin (an anti-microbial agent); allantoin (which stimulates cellular growth and tissue regeneration); and mucilage (which reduces pain and discomfort). Plantago major also has astringent properties giving rise to additional benefits.

The topical sting/bite treatment can further include a natural moisturizer such as Aloe vera. Aloe vera is known for its soothing, moisturizing and healing properties and Aloe vera preparations are commonly used topically for osteoarthritis, burns, sunburns, and psoriasis. Aloe vera gel can be found in hundreds of skin products, including lotions and sun blocks.

The treatment formulation disclosed herein can be applied to the skin with gentle pressure, the natural abrasives operate to help debride the sting site and assist in removing any remaining stinger or other foreign objects imbedded in the skin. The natural antimicrobial agent operates to assist in reducing the incidence of infection and the natural pain reliever and anti-inflammatory assist in reducing the incidence of pain and itch. The moisturizer operates to moisturize the skin and prevent drying.

As contemplated herein, one ingredient may serve more than one purpose in the treatment formulation operating as a natural pain reliever, natural anti-inflammatory or any combination thereof.

Having now described the invention, the construction, the operation and use of preferred embodiments thereof, and the advantageous new and useful results obtained thereby, the new and useful constructions, and reasonable mechanical equivalents thereof obvious to those skilled in the art, are set forth in the appended claims.

What is claimed is:

1. A topical sting/bite treatment compound comprising:
a natural enzyme capable of breaking down proteins;
a natural abrasive capable of debriding or exfoliating an area on the skin;
a natural pain reliever capable or reducing the pain associated with a sting or bite;
a natural anti-inflammatory agent capable or reducing the swelling associated with a pain or bite; and
a natural anti-microbial agent capable of inhibiting an infection resulting from a sting or bite.

2. The topical sting/bite treatment compound of claim 1 wherein said natural enzyme comprises a protease enzyme.

3. The topical sting/bite treatment compound of claim 1 wherein said natural enzyme comprises bromelain.

4. The topical sting/bite treatment compound of claim 1 wherein said natural abrasive is selected from the group consisting of: crushed nut shells; crushed kernels; and crushed seeds.

5. The topical sting/bite treatment compound of claim 1 wherein said natural pain reliever comprises Ocimum sanctum extract.

6. The topical sting/bite treatment compound of claim 1 wherein said natural anti-inflammatory comprises Calendula officinalis extract.

7. The topical sting/bite treatment compound of claim 1 wherein said natural anti-microbial agent comprises Plantago major extract.

8. The topical sting/bite treatment compound of claim 1 wherein said compound further comprises a moisturizer.

9. A method of making a topical sting/bite treatment compound comprising combining effective amounts of: a natural enzyme; a natural abrasive; a natural pain reliever; a natural anti-inflammatory; and a natural anti-microbial agent to create a topical sting/bite treatment compound that neutralizes venom, debrides the wound site, relieves pain, reduces swelling and prevents infection.

10. The topical sting/bite treatment compound of claim 9 wherein said natural enzyme comprises a protease enzyme

11. The topical sting/bite treatment compound of claim 9 wherein said natural enzyme comprises bromelain.

12. The topical sting/bite treatment compound of claim 9 wherein said natural abrasive is selected from the group consisting of: crushed nut shells; crushed kernels; and crushed seeds.

13. The topical sting/bite treatment compound of claim 9 wherein said natural pain reliever comprises Ocimum sanctum extract.

14. The topical sting/bite treatment compound of claim 9 wherein said natural anti-inflammatory comprises Calendula officinalis extract.

15. The topical sting/bite treatment compound of claim 9 wherein said natural anti-microbial agent comprises Plantago major extract.

16. The topical sting/bite treatment compound of claim 9 wherein said compound further comprises a moisturizer.

17. The topical sting/bite treatment compound of claim 16 wherein said moisturizer comprises Aloe vera.
